(19) United States
(12) Patent Application Publication
(10) Pub. No.: US 2021/0274862 A1
(43) Pub. Date: Sep. 9, 2021

(54) TRANSPARENT PATIENT SURGICAL MASK

(71) Applicant: Ray King, Houston, TX (US)

(72) Inventor: Ray King, Houston, TX (US)

(21) Appl. No.: 16/481,525

(22) PCT Filed: Jan. 27, 2018
(86) PCT No.: PCT/US2018/015622

§ 371 Date: Oct. 23, 2019

Related U.S. Application Data
(60) Provisional application No. 62/451,653, filed on Jan. 27, 2017.

(51) Int. Cl. A41D 13/11

(52) U.S. Cl. CPC A41D 13/1161

(57) ABSTRACT

The invention relates to a method and device for creating a transparent face mask to be worn by the patient, to create an effective barrier protecting from the patient's own oronasal flora from contaminating the surgical field and hence risking infection; this mask may be transparent, anti-fogging, and made from semipermeable material(s), with ability to include window(s) (e.g. for entry of endotracheal tube, maintaining a seal between the tube and the mask) and sleeve(s) (e.g. for placement of the intubating blade) and/or other instruments while maintaining qualities of this sterile or clean barrier. The transparent nature of this mask could allow intubation as visualized either directly visually, or in conjunction with a fiberoptic, electrical, or other camera. This mask may be disposable and single-use, and may be used in this sterile or clean setting for medical and other uses.

### CROSS-REFERENCE TO RELATED APPLICATIONS

This Patent Cooperation Treaty International Application claims the benefit and priority of U.S. provisional patent application Ser. No. 62/451,653, filed Jan. 27, 2017, entitled “TRANSPARENT PATIENT SURGICAL MASK”, the contents of which is hereby incorporated by reference in its entirety.

### FIELD OF THE INVENTION

This invention relates generally to devices and methods for maintaining a sterile or enhanced sterility environment in a medical setting, more particularly to devices and methods for sterile or enhanced sterility environment using a face mask for a patient and further particularly to devices and methods for sterile or enhanced sterility environment using a transparent or see-through face mask for a patient, such as a surgical mask used entering, in and/or leaving a sterile operating room environment.

### BACKGROUND OF THE INVENTION

In most surgical procedures presently, all personnel in the presence of sterile surgical instruments (i.e. when the sterile set is open on the table) wear surgical masks to reduce or avoid the risk of respiratory bacteria from causing contamination. Notably absent, however, is the practice of requiring a patient who is undergoing the operation, upon entering the operating room where his/her procedure is to be performed, to wear a similar surgical mask to protect him/herself from his/her respiratory flora (as even his/her own respiratory flora have significant potential for causing an infection by contaminating surgical instruments, e.g. if a patient coughs and bacteria land on surgical instruments to be subsequently used on the same patient's neurosurgical procedure for a brain tumor. A barrier respiratory mask, similar in function to that worn by operating room personnel in the presence of sterile instruments, should be used by the patient upon whom the operation is to be conducted, to help minimize risk of causing infection further. Presently, it is not commonplace to consider such scenario of requiring a patient to wear a mask upon entry into the operating room, though cross-contamination, even from a patient's own body bacteria elsewhere causing an infection if exposed to another of the patient's body parts.

### SUMMARY OF THE INVENTION

The present invention relates to methods and a device for both (1) creation of a barrier between the mouth/nose and the operating room sterile environment, to minimize respiratory or gastro-intestinal bacteria from contaminating surroundings; (2) allowing monitoring of the patient by direct visualization of the nose, mouth and/or surrounding regions by, for example, a transparent or see-through mask or portion thereof, with sleeves, windows and/or other apertures or access points allowing intubation and/or access in a similarly clean manner to nose, mouth, and/or surrounding regions.

These tasks are accomplished using three components: (1) a transparent or see-through mask or portion thereof design that allows visualization of nose, mouth, and/or surrounding structures otherwise behind the mask; (2) sleeves, windows and/or other apertures or access points allowing performance of intubation (e.g. insertion of an intubation blade and endotracheal tube, while maintaining a cleaner-than-present standard of care environment; and (3) ability for the patient to continue respiration via this mask in similar manner as other operating room personnel wearing surgical masks.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 illustrates the transparent mask as placed upon the patient with central hole allowing entry and exit created to accommodate various sizes of endotracheal tubes (ETTs) as it can be stretched to form a seal around the ETT so that air cannot escape (hence may be built from a different, more rigid, or same material as remainder of transparent mask); an elliptical “sleeve” which is not contiguous with the circle for the ETT, but can be folded out or in (much like arms of a long-sleeve shirt) to accommodate an intubating blade (e.g. Macintosh or Miller blade, whether permanent or disposable blade) of varying sizes, so that again the blade doesn't touch the inside of the mouth but the plastic sleeve is maintained as a barrier and used to place the blade used to then displace the tongue for facilitating intubation; the ETT does, however, contact the tongue and throat, with either direct visualization via the non-fogging transparent mask or using a camera (fiberoptic or electrical, as indicated) in conjunction with the ETT for placement;

FIG. 2 illustrates the sleeves with areas which may include apertures in relation to the inside and outside of a patient's mouth; and

FIG. 3 illustrates a transparent mask with interfaces and areas of permeability.

### DETAILED DESCRIPTION OF THE INVENTION

The detailed description set forth below is intended as a description of the presently exemplified methods, devices and compositions provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods and systems for a transparent face mask for the purposes of maintaining a sterile barrier between the patient and the sterile operating room environment, which could be otherwise contaminated by his/her own oronasal respiratory, gastrointestinal, or other bacteria and result in an infection when a similar or another part of his/her body undergoes operation. In general, the face mask may be one-use or otherwise disposable such that cross-contamination from multiple uses or multiple patients does not occur.

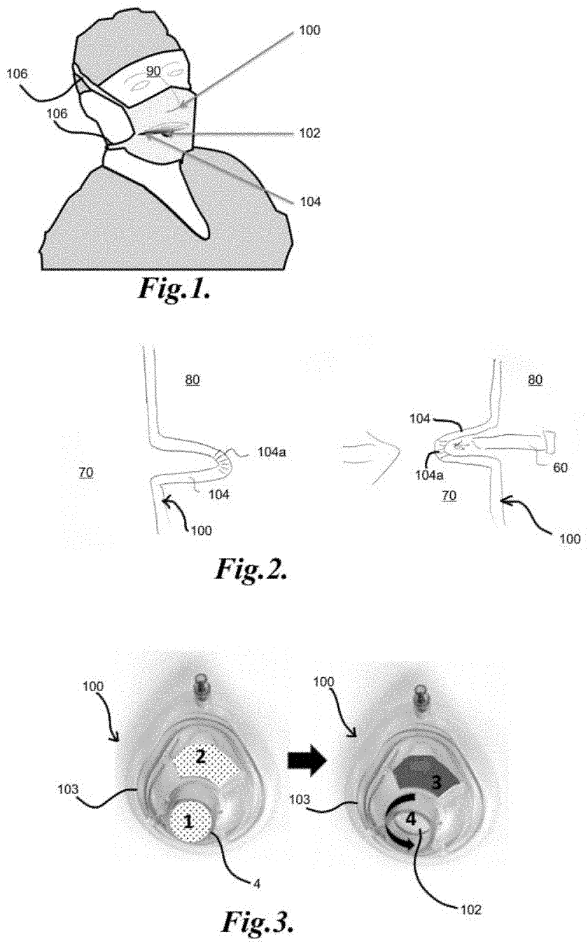

In one aspect of the invention, a device for covering a portion of a patient's face may include a transparent portion which may take up a substantial or nearly all of the device such that the underlying portion of the patient's face may be observable. This may be desirable to enable visualization of the patient's mouth, nose, lips and/or other features, such as to discern distress, communication and/or to visualize interactions with the features. FIGS. 1 and 3 illustrate embodiments of a face mask 100 for covering a portion of a patient's face 90 which may be secured to the patient, such as with straps 106. The face mask 100 or portions thereof be constructed or formed from any appropriate material, such as those which may be transparent and/or see-through. The materials may include, for example, biocompatible polymers, such as permeable, semi-permeable, and/or non-permeable plastic films or combinations thereof, with possibility of meeting N95 or N99 standards as set by FDA (regarding permeability of molecules of given diameter going through the pores). Materials may include, but are not limited to, polypropylene, polystyrene, polycarbonate, polyethylene, polyester, polypropene and/or any other biocompatible plastics in sheet or film format or combinations thereof. In exemplary embodiments, the material may include perforations and/or breathability such that it may be permeable, semi-permeable, and/or non-permeable plastic films or combinations thereof, with possibility of meeting N95 or N99 standards as set by FDA (regarding permeability of molecules of given diameter going through the pores).

In another aspect of the invention, the device for covering a portion of a patient's face may include at least a portion where gas exchange occurs such that the patient may breathe through the regions and/or such that gas exchange facilitates anti-fogging of the transparent portions of the device. While wearing the face mask 100, ventilation may occur (as it does for other operating room personnel), the components of the face mask 100, as well as immediately surrounding regions, may be made from the same or different (e.g. stronger) material of same or varying permeability to air exchange, with anti-fogging quality ideal for part or all of the face mask 100.

For example, the face mask 100, as illustrated in FIGS. 1 and 3, may also be adapted for gas exchange through the material of its construction, such as through perforations or the like or by being breathable, such that the patient may breath through it, while also hindering and/or stopping the movement of contaminants, such as microbes or other pathogens. FIG. 3 further illustrates areas of permeability 1 and 2, such as perforated areas as shown. Portions of the face mask 100 may also be reversibly opened or closed to provide or block gas exchange, as illustrated with port 4, which, as illustrated, may be twisted or turned to switch between open and closed states. Further, the area of permeability 1 may, for example, be a removable cover such that port 4 may serve as an interface or access point, as shown in FIGS. 1 and 3 with an interface 102, for other devices, such as an ambu-bag, ventilator, endotracheal device, video or other stylet/blade for intubation, suction device, and/or any other appropriate device. This may be desirable such that the same face mask 100 may be utilized with a patient without switching out, which may create possibilities of releasing bacteria or other pathogens during the switching.

In a further aspect of the invention, the device may be adapted to provide a seal between the patient's face 90 and the face mask 100 such that gas exchange and/or other ventilation only occurs at designated areas. FIG. 3 illustrates a sealing feature 103 around the perimeter of the face mask 100 that may serve to seal between the face mask 100 and the skin of the patient's face 90. For example, the sealing feature 103 may conform to the skin and provide a seal such that gas exchange only occurs at designated areas, such as the areas of permeability 1 and 2.

In another aspect of the invention, the device may be adapted to provide a barrier between the inside and outside of the face mask 100 to act as a barrier with inserted devices, such as intubating blades (e.g. Macintosh or Miller blade, whether permanent or disposable blade), such that they do not touch the inside of the mouth or throat of the patient. FIGS. 1 and 2 illustrate the face mask 100 with an interface barrier 104 where a portion of the material may be shaped like a pocket or sleeve. FIG. 2 illustrates the inversion of the interface barrier 104 from projecting into the exterior space 80 to the interior space 70 (e.g. the mouth or throat of the patient) when inserting a device, such as the intubating blade 60 as illustrated. Further the interface barrier 104 may include a region for puncturing 104a, such that if desired, an inserted device may be introduced into the interior space 70 by puncturing.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A face mask for a patient comprising:
a covering adapted for covering the mouth and nose of a patient, said covering comprising at least one transparent portion;
a seal adapted to seal against the skin of a patient to prevent gas exchange;
at least one portion adapted to allow gas exchange;
at least one interface portion with an aperture for introducing objects through;
wherein said at least one portion adapted to allow gas exchange is substantially impermeable to pathogens.

2. The face mask of claim 1, wherein said at least one interface portion is reversibly closable.

3. The face mask of claim 1, wherein said face mask is disposable.

4. The face mask of claim 1, wherein said at least one portion adapted to allow gas exchange is perforated or gas permeable.

5. The face mask of claim 1, further comprising an interface sleeve adapted for covering an inserted object to prevent contact with the mouth or throat of a patient.

6. The face mask of claim 5, further comprising a puncturable portion.

7. The face mask of claim 1, further comprising an adapter for interfacing a medical device with said at least one interface portion.

8. The face mask of claim 1, further comprising securing features to hold said covering against said patient's face.
